(19) United States
(12) Patent Application Publication
(10) Pub. No.: US 2019/0290498 A1
(43) Pub. Date: Sep. 26, 2019

(54) ORTHOPEDIC DEVICES

(71) Applicant: Clinch Brace Technologies, LLC., Lehi, UT (US)

(72) Inventors: Tate L. Viehweg, Highland, UT (US); Michael Wagstaff, Farmington, UT (US)

(21) Appl. No.: 15/926,799

(22) Filed: Mar. 20, 2018

(51) Int. Cl. A61F 13/06

(52) U.S. Cl. CPC A61F 13/066; A61F 13/04

(57) ABSTRACT

Orthopedic devices and low temperature shrinking materials used in such devices are described in this application. The low temperature shrinkable materials have a shrink ratio ranging up to about 7:1 at a low temperature ranging from about 100 to about 150° F. Using these lower temperatures to shrink the materials of the orthopedic devices onto the body part of the patient has less risk of injuring the patient than previous materials. The orthopedic devices can contain different sections with higher and lower amounts of the low temperature shrinkable materials. Other embodiments are described.

### FIELD

This application relates generally to orthopedic devices and materials for use in orthopedic devices. More specifically, this application relates to orthopedic devices and low-temperature shrinkable materials used in orthopedic devices.

### BACKGROUND

Orthopedic devices are used to describe medical structures such as casts, splints, supports, braces, and other means utilized to support, immobilize, restrain, protect and position body portions. Orthopedic devices are used in many fields, including the physical medicine and rehabilitation field, general medicine, neurological field. Orthopedic devices are also used to prevent recurrence of previous disabilities, and to prevent discomfort and subsequent disability.

Different types of known orthopedic devices have various uses. For example, plaster casts are commonly used for the treatment of fractures. However, plaster casts can take hours to harden, are excessively heavy, commonly have poor compression strength, are readily crushed or broken, and characteristically have poor resistance to water. As another example, ankle injuries are commonly treated with ankle supports in the form of ankle wraps and/or athletic tape. However, wrapping and/or taping ankles is notoriously a slow cumbersome process, expensive, inherently inconsistent, and requires the talent of a skilled person. These are a few of many examples demonstrating the need for orthopedic devices that are practical, suitable for their intended purpose, and capable of being formed in a customizable, generally consistent, convenient, effective, and timely manner.

### SUMMARY

This application relates to orthopedic devices and low temperature shrinkable materials used in such devices. The low temperature shrinkable materials have a shrink ratio ranging up to about 7:1 at a low temperature ranging from about 100 to about 150° F. Using these lower temperatures to shrink the materials of the orthopedic devices onto the body part of the patient has less risk of injuring the patient than previous materials. The orthopedic devices can contain different sections with higher and lower amounts of the low temperature shrinkable materials.

In some configurations, the orthopedic devices comprise a tubular body forming an interior surface and an exterior surface and including: a leg portion including a first opening; a foot portion connected to said leg portion and including a second opening, said second opening positioned in a back portion of said foot portion and configured to receive a heel of the user; a thickness extending between said interior surface and said exterior surface, said thickness comprising a first thickness in side portions of said tubular body configured to provide lateral support to the ankle and a second thickness in a front portion of said tubular body, said first thickness being greater than said second thickness; and a low temperature shrinkable material having a shrink ratio ranging up to about 7:1 at a temperature ranging from about 100 to about 150 degrees Fahrenheit; where the tubular body moves between a first position, wherein said tubular body is configured to at least partially receive and at least partially surround at least a portion of the ankle, and a second position, wherein one or more portions of at least one of said leg portion or said foot portion are configured to generally conform to and at least partially restrict movement of the at least a portion of the ankle, and wherein said tubular body is configured to move from said first position to said second position when heated at said temperature.

In other configurations, the orthopedic devices comprise a first section containing up to 10 vol % of a low temperature shrinkable material having a shrink ratio ranging up to about 7:1 at a low temperature from about 100 to about 150° F.; a second section containing up to 10 vol % of a low temperature shrinkable material; a middle section between the first and second sections, the middle section configured to strengthen a part of the body needing support and containing a first portion containing more than about 90 vol % of a low temperature shrinkable material; and a second portion containing about 60 to about 90 vol % of a low temperature shrinkable material; wherein the middle section can move between a first position and a second position, the first position configured to partially receive and partially surround that body part and the second position configured to generally conform to and partially restrict movement of that body part, and wherein the middle section is configured to move from the first position to the second position when heated at the low temperature.

In other configurations, the orthopedic devices comprise a first section containing less than about 50 vol % of a low temperature shrinkable material having a shrink ratio ranging up to about 7:1 at a low temperature from about 100 to about 150° F.; a second section larger than the first section, the second section configured to strengthen a part of the body needing support and containing more than about 50 vol % of the low temperature shrinkable material; and wherein the orthopedic device can move between a first position and a second position, the first position configured to partially receive and partially surround that body part and the second position configured to generally conform to and partially restrict movement of that body part, and wherein the orthopedic device is configured to move from the first position to the second position when heated at the low temperature.

### BRIEF DESCRIPTION OF THE DRAWINGS

The following description can be better understood in light of Figures herein which show various embodiments and configurations of the communication systems. Together with the following description, the Figures demonstrate and explain the principles of the structures, methods, and principles described herein. In the drawings, the thickness and size of components may be exaggerated or otherwise modified for clarity. The same reference numerals in different drawings represent the same element, and thus their descriptions will not be repeated.

FIG. 1A illustrates a perspective view of an ankle support according to some embodiments;

FIG. 1B illustrates some embodiments of a back elevation view of the ankle support shown in FIG. 1A;

FIG. 1C illustrates a cross-sectional view of the ankle support shown in FIG. 1B taken along line 1C-1C;

FIG. 1D illustrates a cross-sectional view of the ankle support shown in FIG. 1C taken along line 1D-1D;

FIG. 2 depicts a bottom view of other embodiments of an ankle support;

FIG. 3 shows a side view of other embodiments of an ankle support;

FIG. 4 depicts a back view of yet other embodiments of an ankle support;

FIG. 5 shows a side view of other embodiments of an ankle support; and

FIG. 6 illustrates a side view of still other embodiments of an ankle support.

In addition, as the terms on, disposed on, attached to, connected to, or coupled to, etc. are used herein, one object (e.g., a material, element, structure, member, etc.) can be on, disposed on, attached to, connected to, or coupled to another object—regardless of whether the one object is directly on, attached, connected, or coupled to the other object or whether there are one or more intervening objects between the one object and the other object. Also, directions (e.g., on top of, below, above, top, bottom, side, up, down, under, over, upper, lower, lateral, orbital, horizontal, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

### DETAILED DESCRIPTION

The following description supplies specific details in order to provide a thorough understanding. Nevertheless, the skilled artisan will understand that the described devices can be implemented and used without employing these specific details. Indeed, the described devices and methods can be placed into practice by modifying the described systems and methods and can be used in conjunction with any other apparatus and techniques conventionally used in the industry.

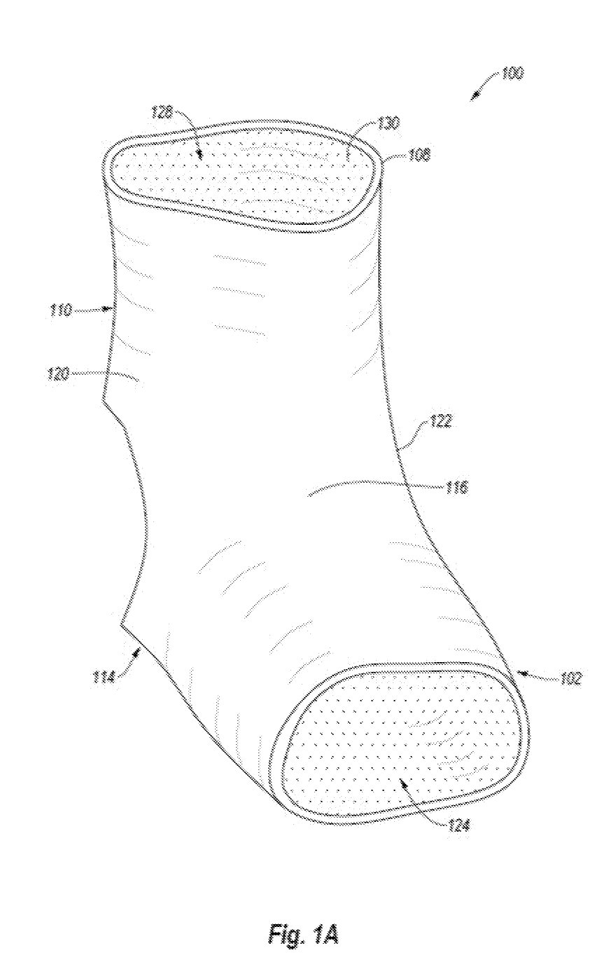

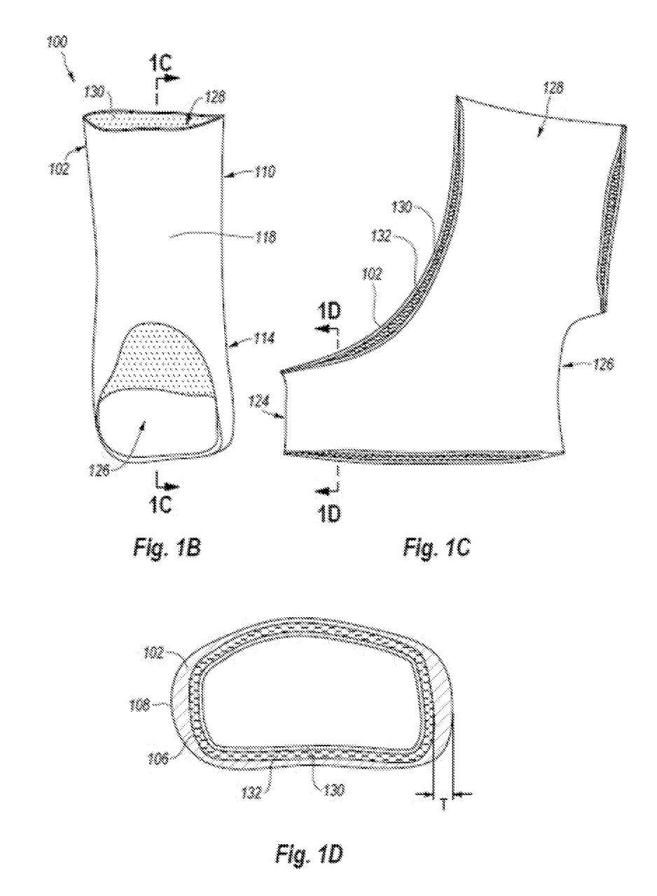

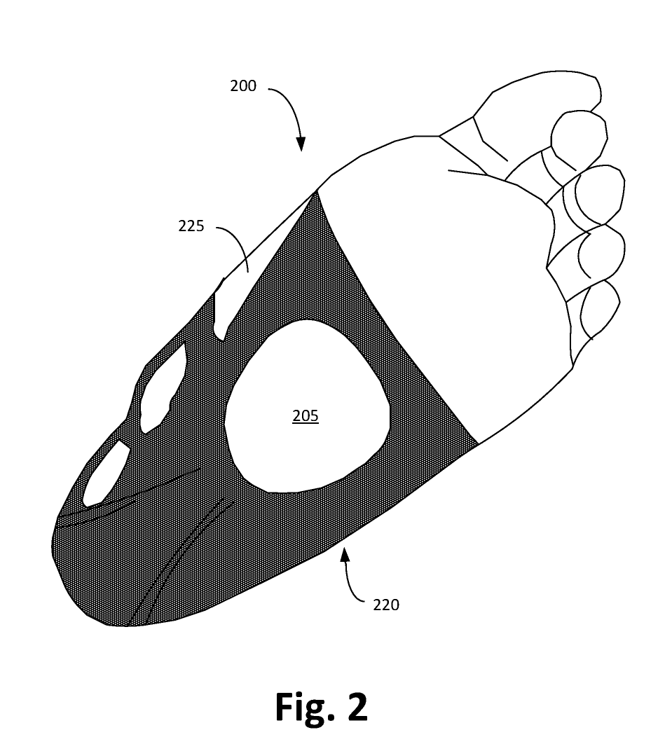

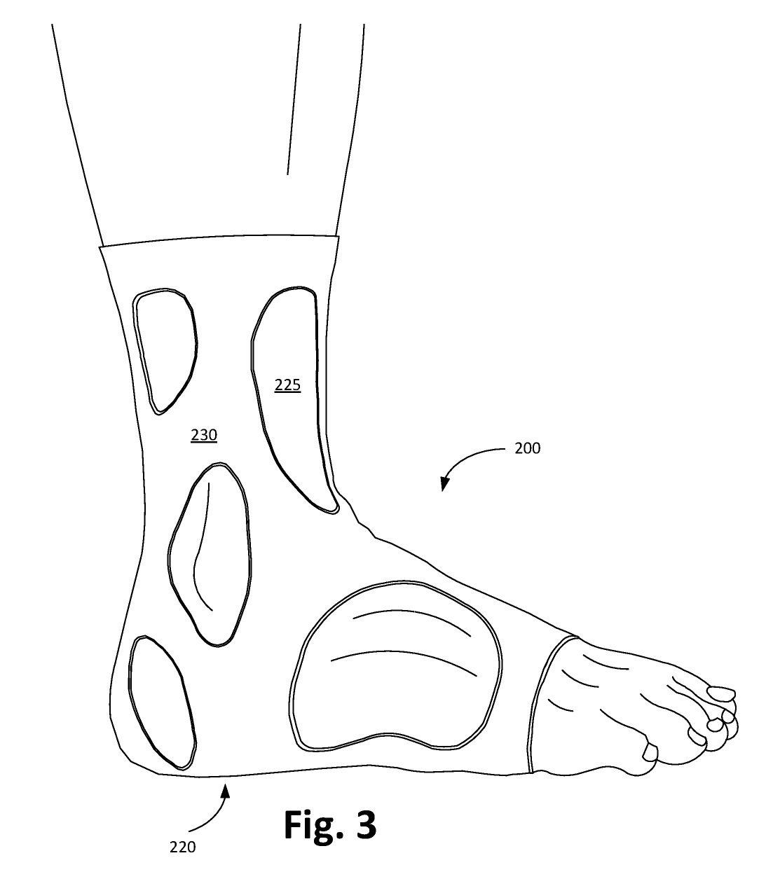

Some embodiments of the orthopedic devices have been described in U.S. Pat. No. 8,979,783 (the '783 Patent), the entire disclosure of which is incorporated herein by reference. The '783 Patent describes some embodiments of orthopedic devices comprising ankle braces, knee braces, and finger braces. As depicted in FIGS. 1A through 1D, the ankle brace 100 contained a tubular body 102 forming an interior surface 106 and an exterior surface 108 (shown in FIG. 1D). The ankle brace 100 may include a liner 130 configured to help secure the ankle brace 100 to the foot, ankle, and/or leg of the user, protect the user, support the foot, ankle, and/or leg, and/or provide a comfortable fit. The liner 130 may include a plurality of small perforations for enhanced flexibility and/or ventilation of the ankle brace 100. The body 102 may be at least partially defined by a front portion 116, a back portion 118, a first side portion 120, and a second side portion 122 and may include a foot portion 114 and a leg portion 110. The foot portion 114 may be sized and configured to receive and substantially surround a foot and an ankle. As shown, the foot portion 114 may also include a first opening 124 configured to allow toes of the user to extend therethrough and a second opening 126 on the back portion 118 configured to receive a heel of a user. In other embodiments, the first opening 124 and/or the second opening 126 may be omitted. The leg portion 110 may be sized and configured to at least partially surround and/or cover a lower portion of a leg. The leg portion 110 may include a third opening 128 sized and configured to allow the foot and the lower portion of the leg to be positioned within the body 102. In an embodiment, the body 102 may comprise a single member. In other embodiments, the body 102 may comprise two, three, four, or any other suitable numbers of members. For example, the leg portion 110 and the foot portion 114 may be attached to one another via stitching, adhesives, fasteners, combinations thereof, or other suitable attachment means. In addition, the leg portion 110 and the foot portion 114 may be attached to one another via two or more connecting members or stirrups. The ankle brace 100 may be configured to be used on a user's right ankle or left ankle.

Referring now to FIGS. 1C and 1D, the ankle brace 100 may include the body 102, the liner 130, and an intermediate layer 132. As shown, the body 102 may have a thickness T extending between the interior surface 106 and the exterior surface 108 of the body 102. In an embodiment, the body 102 has a thickness T of about 0.5-millimeters (mm) to 5-mm. In other embodiments, the thickness T may be about 0.5-mm to 30-mm; 2-mm to 20-mm; or 3-mm to 10-mm. The thickness T may be generally thinner to provide a more lightweight construction of the body 102 or the thickness T may be generally thicker to provide a more robust construction of the body 102. In other embodiments, the thickness T may be either greater or smaller. In an embodiment, the thickness T of the body 102 may be generally uniform. In other embodiments, the thickness T of the body 102 may vary based on the anatomy of the user. For example, the body 102 may have a greater thickness T about the ankle and a lesser thickness T about the foot of the user. Such a configuration of the body 102 may provide additional support to the ankle and/or may allow the ankle brace 100 to be more comfortably positioned within a shoe. In addition, the foot portion 114 of the body 102 may include a greater thickness T along at least a portion of the bottom of the foot to provide additional cushioning to the user and/or arch support.

In yet other embodiments, the thickness T of the body 102 may vary to influence stiffness and/or flexibility of the ankle brace 100 in different locations. Controlling the flexibility and/or stiffness of the ankle brace 100 in different locations may allow the ankle brace to maintain its structural integrity while providing desired support, protection, positioning, and/or restraint. For example, as shown in FIG. 1D, the first side portion 120 and the second side portion 122 of the body 102 may have a greater thickness T than at least the front portion 116 of the body 102. Such a configuration may provide additional lateral support to the user and/or may substantially restrict abnormal eversion and/or inversion while allowing dorsiflexion and/or plantar flexion. The thickness T of the body 102 may also be configured to substantially immobilize the ankle. For example, the body 102 may have a greater thickness T about the front, back and sides of the ankle of the user such that the ankle brace 100 provides sufficient stiffness and/or rigidity about the ankle to substantially immobilize the ankle. Accordingly, the ankle brace 100 may be configured for use in a variety of applications including sports, medical treatment, emergency response, or other suitable applications.

In an embodiment, the liner 130 may be attached to the interior surface 106 of the body 102 along at least a portion of one or more edges of the liner 130. In other embodiments, the liner 130 may be separate from the body 102 and/or the ankle brace 100. For example, the liner 130 may be configured as a protective sock and may be placed on the user prior to placement of the body 102 and/or the ankle brace 100 on the user. In yet other embodiments, the liner 130 may be omitted. The liner 130 may be sized and/or configured to provide a comfortable fit for the user as well as stability. For example, the liner 130 may be comprised of a soft, compressible material such as polyurethane foam, polyethylene foam, microspheres contained in a lubricant matrix, other gels and foams, pneumatics, combinations thereof, or other suitable materials. In other embodiments, the liner 130 may comprise a thermoplastic material that once shrunk, remains relatively flexible and comfortable. Similar to the body 102, the liner 130 may have a thickness that is substantially uniform or varies based on anatomy, desired flexibility, and/or a desired stiffness of the liner 130. The liner 130 may also be sized and/or configured to at least partially protect the user from the treatment. For example, the treatment may comprise a heat treatment and the liner 130 may be configured as a protective layer to generally insulate the user from the heat treatment. In another embodiment, the treatment may comprise a chemical treatment and the liner 130 may be configured to provide an impermeable barrier to substantially protect the user from the chemical treatment.

The intermediate layer 132 may be interposed between the interior surface 106 of the body 102 and the liner 130. Similar to the liner 130, the intermediate layer 132 may be sized and/or configured to provide a comfortable fit, stability, and/or protection to a user from the treatment. For example, the intermediate layer 132 may have a thickness preconfigured to provide a physical barrier between the user and the treatment. In another embodiment, the intermediate layer 132 may comprise one or more insulation materials configured to at least partially protect the user from a treatment comprising a heat treatment. For example, the intermediate layer 132 may comprise polyurethane, elastomeric foam, mineral wool, phenolic foam, polystyrene, combinations thereof, of any other suitable insulation materials. In yet other embodiments, the intermediate layer 132 may be configured to dissipate heat before heat from a heat treatment reaches the liner 130 and/or the user. For example, the intermediate layer 132 may comprise water, air, gels, combinations, thereof, or any suitable heat dissipating material. Moreover, similar to the body 102, the thickness of the intermediate layer 132 may be substantially uniform or may vary based on anatomy, desired flexibility, and/or a desired stiffness of the liner 130.

The ankle brace 100 contains one or more features configured to selectively move the ankle brace 100 between a receiving position, wherein the ankle brace 100 may receive and/or generally surround the leg, the ankle, and/or the foot, and a supporting position, wherein one or more portions of the ankle brace 100 substantially conforms to and/or at least partially restrict movement of the ankle in one or more directions. For example, the body 102 may be made from a variety of different shrinkable materials that are configured to shrink the ankle brace 100 into the receiving position in response to a treatment. Shrinkable materials are materials capable of being reduced in size in response to a treatment. Specifically, the tubular body 102 may comprise one or more thermoplastic materials, polyolefin, polyolefin elastomer, nylon, neoprene, silicone rubber, woven fabric, combinations thereof, or other suitable shrinkable materials. In an embodiment, the shrinkable materials may be uniform throughout the body 102. In other embodiments, the shrinkable materials may vary at different locations on the body 102. For example, the first side portion 120 and the second side portion 122 in the area of the user's ankle may comprise a shrink material that in the supporting position becomes generally stiff or rigid to at least partially restrict abnormal eversion and/or inversion of the ankle. In addition, the front portion 116 in the area of a user's lower leg portion may comprise a shrink material that in the supporting position remains generally flexible to generally allow dorsiflexion and/or plantar flexion. This patent described similar braces that could be configured for the knee, finger, or other parts of the body that needed to be supported.

As described in the '783 Patent, the braces contain shrinkable materials that may be configured to control the shrinkage of portions of the brace. The selected portions of the brace could be shrunk by a treatment process, including a heat treatment process using a shrink temperature. The shrink temperature is the minimum temperature at which a shrinkable material shrinks. Unfortunately, many materials that were used in the heat treatment process of the '783 Patent had a high shrinkage temperature that can injure the user (i.e., the patient) or even the medical personnel placing the device on the patient.

Accordingly, in some embodiments, the ankle brace can contain materials with a low shrink temperature. In these embodiments, the shrink temperature can be the same or lower than the shrink temperature of the materials described in the '783 Patent. Accordingly, in some embodiments the shrink temperature can range from about 100 to about 150 degrees Fahrenheit. In yet other embodiments, the shrink temperature can range from about 110 to about 130 degrees Fahrenheit. In yet other embodiments, the shrink temperature can be any combination or sub-range of these degrees.

The low temperature shrinkable materials may be configured to control the amount of shrinkage of the ankle brace 100. For example, they may have a shrink ratio between about 7:1 to 1.1:1; 6:1 to 1.3:1; 5:1 to 1.5:1; or even 4:1 to 2:1. The shrink ratio is the quantitative relation between the initial size of the body 102 and the shrunken size of the body 102. In yet other embodiments, the shrink ratio can be any combination or sub-range of these ratios. In other embodiments, including those described below, the ankle brace 100 may include shrinkable materials having different shrink ratios in different locations on the ankle brace 100.

In these embodiments, these materials also exhibit other features that can be useful for the brace. One of these features is their strength. In some embodiments, the tensile strength of the materials can range from about 100 to about 200 MPa as measured by the D2256 ASTM method. In other embodiments, the tensile strength of the materials can be about 145 MPa. As well, in some embodiments, the break strength of the materials can range from about 2 to about 20 lbs. as measured by the D2256 ASTM method. In other embodiments, the break strength of the material can be about 4.3 lbs. In yet other embodiments, the material strength can be any combination or sub-range of these amounts.

Another feature that can be useful for the braces is the density of the material. In some embodiments, the materials can have a density of about 200 deniers or less. In other embodiments, the materials have a density ranging from about 150 deniers to about 200 deniers.

In some embodiments, these materials can comprise any thermoplastics material such as PET (polyethylene terephthalate), PP (polypropylene), PLA (polylactic acid), or combinations of these materials. In other embodiments, these materials can include amorphous PLA, crystalline PLA, or combinations thereof. The materials may comprise un-oriented yarns, partially oriented yarns, fully oriented yarns, or combinations thereof. These yarns can be woven or knit into the desired configuration and, therefore, are different than the materials described in the '783 Patent which are formed (i.e., extruded) as a continuous sheet. These materials are also stronger as a result of the weave or knit pattern.

Other configurations can be used for the braces. For example, these materials may allow an ankle brace 100 to be customized and/or conformed to a specific user in a minimal amount of time. As well, less materials need be used in the brace to provide the needed support to the desired portion of the patient's body, as shown in FIGS. 2-6.

FIGS. 2-3 show some embodiments of these different configurations that can be used for these braces. FIG. 2 shows a bottom view and FIG. 3 shows a side view of these embodiments. In FIGS. 2-3, the brace 200 can be configured that it does not need to cover the entire portion of the foot or ankle. In other words, unlike the configuration of the braces shown in the '783 Patent, the brace does not need to be made continuous, but may contain openings or hollow portions.

In FIG. 2, the brace 200 contains an opening 205 (or bottom opening) that does not contain any of the material used for the brace. The opening 205 is surrounded by the bottom portion 220 of the brace 200. The opening 205 can have any shape or size provided that the rest of the brace 200 provides the needed support for the ankle of the patient. So while the first opening 205 is shown substantially circular in FIG. 2, it can any other shape such as triangular, square, rectangular, polygonal, etc., or even any irregular shape. In other configurations, there can be multiple bottom openings 205 in the brace 200.

In FIG. 3, the brace 200 has one or more side openings 225 surrounded by the side portion 230 of the brace. Like the opening(s) 205, the opening(s) 225 can have any shape or size provided that the rest of the brace 200 provides the needed support for the ankle of the patient. So while the openings 225 are shown substantially circular in FIG. 2, they can any other shape such as triangular, square, rectangular, polygonal, etc., or even any irregular shape. And the shapes from one of the openings 225 to the next opening 225 can change. As well, the sizes of the openings 225 can be substantially similar or can be different.

The braces 200 shown in FIGS. 2-3 can have any of features of the braces 100 described above. As well, the braces shown in the embodiments depicted in FIGS. 2-3 can have any of the features of the braces described in the '783 Patent. For example, the brace 200 can contain more than about 50 vol %, more than about 60 vol %, more than about 70 vol %, more than about 80 vol %, more than about 90 vol %, more than about 95 vol %, or any combination or sub-range of these amounts, of the lower temperature shrinkable materials.

In FIGS. 2-3, the openings 205 and 225 are shown as containing no material. In other embodiments, though, these openings can contain materials that provide a lower amount of support than the rest of the material used for the brace 200 so that the brace 200 is made of multiple sections with different materials. In these configurations, the strongest materials are present where they are needed most and the openings are not really an opening, but merely an area where weaker materials can be used. In other words, the configurations shown in FIGS. 2 and 3 can have any combination of lower temperature shrinkable materials since they these materials are stronger and can be located in the area that runs with the anatomy and accordingly bolsters and/or supports the anatomical joint. Therefore the pattern(s) of the lower temperature shrinkable materials can mimic the desired part of the anatomy. In these configurations, the openings can contain less than about 50 vol %, less than about 40 vol %, less than about 30 vol %, less than about 20 vol %, less than about 10 vol %, less than about 05 vol %, or any combination or sub-range of these amounts, of the low temperature shrinkable materials.

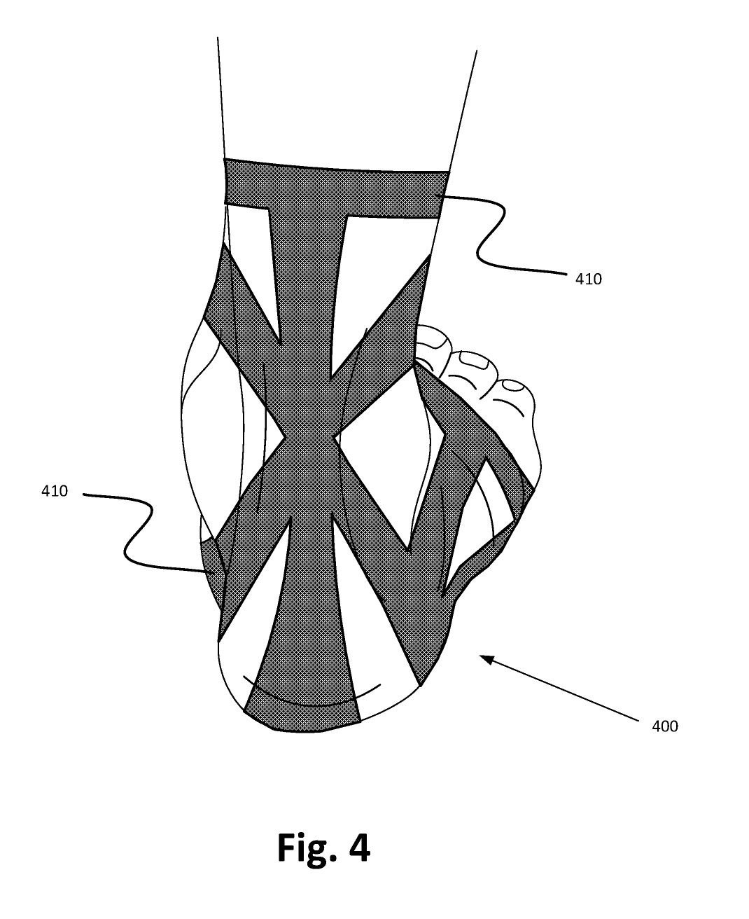

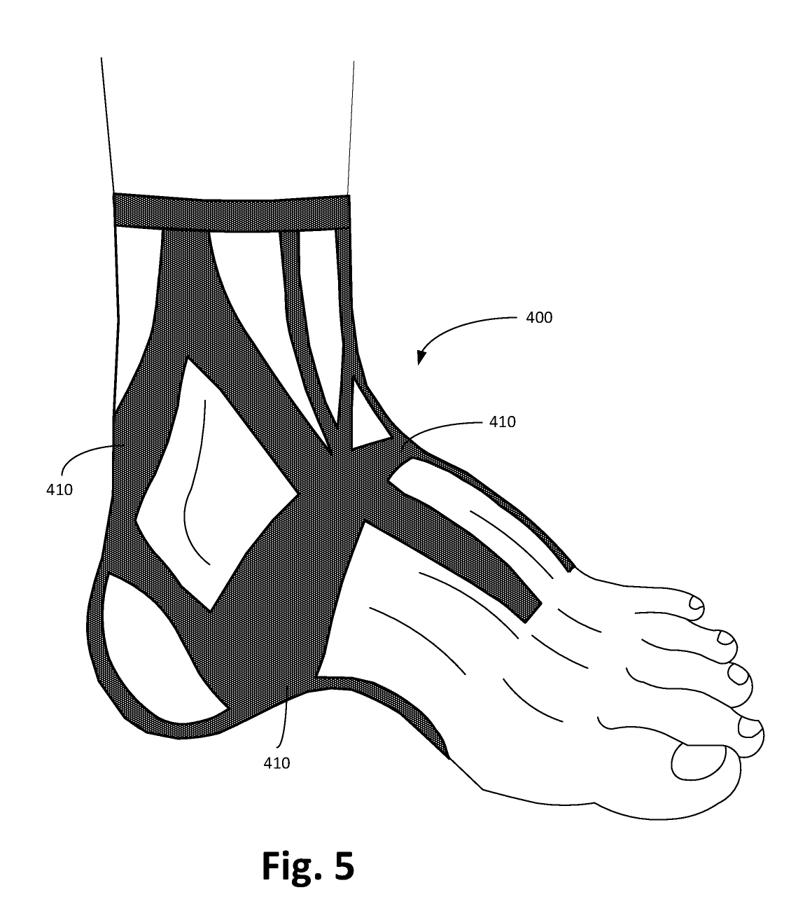

FIGS. 4-5 show additional embodiments of different configurations that can be used for these braces. FIG. 4 shows a back view and FIG. 5 shows a side view of brace 400. In FIGS. 4-5, the brace 400 can be configured so that rather than containing openings, it comprises strips (or struts) of material that provide the needed support to the ankle or other desired part of the patient. In these configurations, the brace 400 can comprise strips (or ribbons) 410 of materials that together provided the needed support to the ankle. The strips can be given various configurations of lengths, width, and thicknesses that are needed for the specific patient and/or the specific injury of the patient. And while the strips 410 are shown as roughly rectangular in shape, they can be given any other shape including circular, square, polygonal, irregular, etc. . . . . They can also be placed on any part(s) of the foot and/leg of the patient to provide the needed support. In these embodiments, the stronger materials are present where there is the most need for the desired portions of the anatomy.

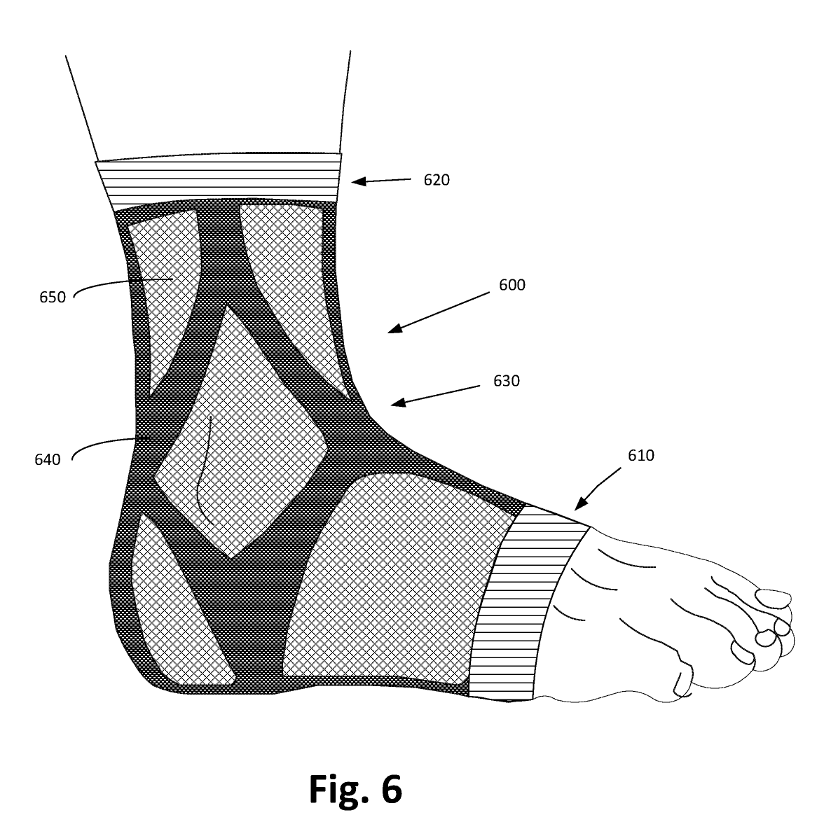

FIG. 6 shows yet other embodiments of different configurations that can be used for these braces. In FIG. 6, the brace 600 can be configured so that different portions of the braces contain different amounts of the materials described herein, including the low temperature shrinkable materials. The brace 600 contains a first portion (or bottom portion) 610 of the brace that is situated on the bottom of the brace (i.e., near the toes). The brace 600 also contains a second portion (or top portion) 620 that is situated on the top of the brace (i.e., near the calf). The brace 600 also contains a third middle portion 630 that is situated between the top and bottom portions. The middle portion provides the majority of the supporting function of the brace 600.

The top and bottom portions of the brace are made of a minimal amount of the low temperature shrinkable materials. In some configurations, the top and bottom portions contain anywhere from 0 to about 10 vol % of the low temperature shrinkable materials. The top and bottom portions provide less of a support function than the middle portion. Therefore, they need to shrink less than the middle portion and only need to contain a small amount of the low temperature shrinkable materials.

The middle portion 630 of the brace 600 provides most of the support function and therefore needs to have a higher concentration of the low temperature shrinkable materials. But it does not need to contain a homogenous concentration of the low temperature shrinkable materials since some parts of the middle portion provide a higher concentration than other parts. So those parts of the middle portion that provide more support function contain a higher amount of the low temperature shrinkable material.

In the configurations shown in FIG. 6, the middle portion contains a first part 640 that contains a higher amount of the low temperature shrinkable material and a second part 650 that contains a lower amount. In some embodiments, the first part can contain more than about 90 vol % of the low temperature shrinkable material. In other embodiments, the first part can contain about 95 to about 100 vol % of the low temperature shrinkable material. In yet other embodiments, the first part can contain any combination or sub-range of these amounts. In some embodiments, the second part can contain about 60 to about 90 vol % of the low temperature shrinkable material. In other embodiments, the second part can contain about 70 to about 80 vol % of the low temperature shrinkable material. In yet other embodiments, the second part can contain any combination or sub-range of these amounts.

Although the orthopedic devices and systems described above have been discussed in the context of ankle braces. In other embodiments, the orthopedic devices and systems disclosed herein are not limited to such use and may be used for many different applications, if desired, without limitation as described in the '783 Patent. Thus, such orthopedic devices and systems are not limited for use with ankle braces and may be used with various other braces and supports, without limitation. Indeed, the orthopedic devices and systems described above are not limited to being used on human patients, but also on animals. As well, while the low temperature shrinkable materials are described with reference to FIGS. 1A-1D, they can be used in any of the embodiments described in FIGS. 2-6.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. An orthopedic device, comprising:
a tubular body forming an interior surface and an exterior surface and including:
a leg portion including a first opening;
a foot portion connected to said leg portion and including a second opening, said second opening positioned in a back portion of said foot portion and configured to receive a heel of the user;
a thickness extending between said interior surface and said exterior surface, said thickness comprising a first thickness in side portions of said tubular body configured to provide lateral support to the ankle and a second thickness in a front portion of said tubular body, said first thickness being greater than said second thickness; and
a low temperature shrinkable material having a shrink ratio ranging up to about 7:1 at a shrink temperature ranging from about 100 to about 150 degrees Fahrenheit;
said tubular body being moveable between a first position, wherein said tubular body is configured to at least partially receive and at least partially surround at least a portion of the ankle, and a second position, wherein one or more portions of at least one of said leg portion or said foot portion are configured to generally conform to and at least partially restrict movement of the at least a portion of the ankle, and wherein said tubular body is configured to move from said first position to said second position when heated at said temperature.

2. The orthopedic device of claim 1, wherein a side portion of said foot portion is substantially stiff in said second position and a front portion of said foot portion is substantially flexible in said second position.

3. The orthopedic device of claim 1, wherein said tubular body is configured to restrict at least one of abnormal eversion or abnormal inversion of the ankle in said second position.

4. The orthopedic device of claim 1, wherein said tubular body is substantially stiff in said second position.

5. The orthopedic device of claim 1, wherein the low temperature shrinkable material comprises polyethylene terephthalate, polypropylene, polylactic acid, or combinations thereof.

6. The orthopedic device of claim 1, wherein the low temperature shrinkable material comprises amorphous polylactic acid, crystalline polylactic acid, or combinations thereof.

7. The orthopedic device of claim 1, further comprising a liner proximate said interior surface of said tubular body, said liner being configured to substantially protect at least one of the ankle or the foot from the treatment.

8. The orthopedic device of claim 1, further comprising a liner and an intermediate layer interposed between said liner and said interior surface of said tubular body, said intermediate layer being configured to substantially insulate at least one of the ankle or foot from the treatment, wherein the treatment comprises a heat treatment.

9. The orthopedic device of claim 1, wherein the shrink temperature can range from about 110 to about 130 degrees Fahrenheit.

10. An orthopedic device, comprising:
a first section containing up to 10 vol % of a low temperature shrinkable material having a shrink ratio ranging up to about 7:1 at a shrink temperature from about 100 to about 150° F.;
a second section containing up to 10 vol % of a low temperature shrinkable material;
a middle section between the first and second sections, the middle section configured to strengthen a part of the body needing support and comprising:
a first portion containing more than about 90 vol % of a low temperature shrinkable material; and
a second portion containing about 60 to about 90 vol % of a low temperature shrinkable material;
wherein the middle section can move between a first position and a second position, the first position configured to partially receive and partially surround that body part and the second position configured to generally conform to and partially restrict movement of that body part, and
wherein the middle section is configured to move from the first position to the second position when heated at the low temperature.

11. The orthopedic device of claim 10, wherein the shrink temperature ranges from about 110 to about 130 degrees Fahrenheit.

12. The orthopedic device of claim 10, wherein a first portion of the body part is substantially stiff in the second position and a second portion of the body part is partially flexible in the second position.

13. The orthopedic device of claim 10, wherein the first portion of the middle section can contain about 95 vol % or more of the low temperature shrinkable material.

14. The orthopedic device of claim 10, wherein the second portion of the middle section can contain about 70 to about 80 vol % of the low temperature shrinkable material.

15. The orthopedic device of claim 10, wherein the body part is an ankle.

16. The orthopedic device of claim 10, wherein the low temperature shrinkable material comprises amorphous polylactic acid, crystalline polylactic acid, or combinations thereof.

17. An orthopedic device, comprising:
a first section containing less than about 50 vol % of a low temperature shrinkable material having a shrink ratio ranging up to about 7:1 at a shrink temperature from about 100 to about 150° F.;
a second section larger than the first section, the second section configured to strengthen a part of the body needing support and containing more than about 50 vol % of the low temperature shrinkable material; and
wherein the orthopedic device can move between a first position and a second position, the first position configured to partially receive and partially surround that body part and the second position configured to generally conform to and partially restrict movement of that body part, and
wherein the orthopedic device is configured to move from the first position to the second position when heated at the low temperature.

18. The orthopedic device of claim 17, wherein the shrink temperature ranges from about 110 to about 130 degrees Fahrenheit.

19. The orthopedic device of claim 17, wherein the first section is configured as an opening in the second section.

20. The orthopedic device of claim 17, wherein the opening is configured as substantially circular in shape.
